(19) United States
(12) Patent Application Publication
(10) Pub. No.: US 2018/0318228 A1
(43) Pub. Date: Nov. 8, 2018

(54) METHOD FOR A SLOW RELEASE OF DRUGS FROM ORALLY DISSOLVING CAPSULES

(71) Applicants: Hemant N. Joshi, Parsippany, NJ (US); Amitkumar N. Lad, Dumont, NJ (US)

(72) Inventors: Hemant N. Joshi, Parsippany, NJ (US); Amitkumar N. Lad, Dumont, NJ (US)

(21) Appl. No.: 16/004,896

(22) Filed: Jun. 11, 2018

Related U.S. Application Data
(63) Continuation of application No. 15/589,655, filed on May 8, 2017.

(51) Int. Cl. A61K 9/48; A61K 31/566; A61K 36/534; A61K 36/61; A61K 36/9066; A61K 31/137; A61K 31/675; A61K 31/366; A61K 31/453; A61K 9/00; A61K 31/192

(52) U.S. Cl. CPC A61K 9/4808; A61K 31/566; A61K 36/534; A61K 36/61; A61K 36/9066; A61K 31/137; A61K 31/675; A61K 31/192; A61K 31/366; A61K 9/4858; A61K 9/4866; A61K 31/453; A61K 9/4816; A61K 9/006

(57) ABSTRACT

A method for a slow-release of drugs from orally dissolving capsule has been described. It releases medicaments in the mouth cavity up to 1 hour. The medicaments may have a therapeutic effect in the mouth cavity and a portion of the drug is delivered to stomach. Thus, the medication may have a local action in the oral cavity and/or have a systemic effect. Mainly, the orally dissolving capsule is administered without an aid of water. Also, large capsules such as size “000” can be administered allowing delivery of large amounts (800 to 1600 mg) of medicament. The medicament can be absorbed through mouth cavity (oral transmucosal absorption) to blood stream bypassing hepatic first pass metabolism. In some instances, a drug(s) is incorporated in the ODC empty shell matrix and no drug composition is filled inside the capsule shell core.

### RELATED APPLICATION

This application is a continuation of and claims the benefit of the co-pending U.S. patent application Ser. No. 15/589,655 filed May 8, 2017, titled “Drug Delivery with Orally Dissolving Capsules”.

### FIELD OF THE INVENTION

The present invention relates to method of slow-release of medicaments in the mouth cavity for local action or to gastro-intestinal tract for a systemic action using orally dissolving capsules (ODCs). Formulation contains a pharmaceutical active ingredient(s), plasticizer, water-soluble flavor, sweetener, preservatives and other excipients. The ODCs allow drug administration without water, and it may allow oral transmucosal absorption thereby helping to reduce hepatic first-pass effect.

### BACKGROUND OF THE INVENTION

Capsules are one of the most commonly pharmaceutical dosage forms, which are easy to manufacture. Capsules are made from aqueous solution of a gelling agent such as animal protein or plant polysaccharide or their derivatives. Capsules are generally of two types: hard and soft capsules. Hard capsules as name suggests are hard for feel and soft being soft.

Many specific constituents have been used to form the shell of capsule. One basic component is film forming material or matrix such as gelatin, hydroxylpropyl methylcellulose (HPMC), starch, cellulose or other polymer materials.

Capsules are readily soluble in water at 37° C. Types of materials for filling into hard gelatin capsules are dry solids such as powders, pellets, granules or semisolids such as suspensions, pastes or liquids such as non-aqueous liquids.

There are various sizes of hard capsule shells available ranging from sizes ‘000’ to ‘5’ (higher the number, smaller is the dose volume), the most commonly used are sizes ‘0’ and ‘1’.

A hard-capsule size chart is shown in Table 1. These values may vary for each capsule manufacturer.

TABLE 1
Details on various sizes of capsules including the amount of powder that can be filled.

| | Size | | | | | | | | | |
|---|---|---|---|---|---|---|---|---|---|---|
| | 000 | 00el | 00 | 0el | 0 | 1 | 2 | 3 | 4 | 5 |
| Volume, mL | 1.37 | 1.02 | 0.95 | 0.78 | 0.68 | 0.50 | 0.37 | 0.30 | 0.21 | 0.10 |
| Weight empty | 158.1 | 128 | 115.7 | 99.7 | 89.8 | 71.8 | 58.1 | 48.6 | 37.5 | 24.1 |
| capsule (mg) | | | | | | | | | | |
| Density (g/cc) | Powder weight in mg | | | | | | | | | |
| 0.3 | 411 | 306 | 285 | 234 | 204 | 150 | 111 | 90 | 63 | 30 |
| 0.4 | 548 | 408 | 380 | 312 | 272 | 200 | 148 | 120 | 84 | 40 |
| 0.5 | 685 | 510 | 475 | 390 | 340 | 250 | 185 | 150 | 105 | 50 |
| 0.6 | 822 | 612 | 570 | 468 | 408 | 300 | 222 | 180 | 126 | 60 |
| 0.7 | 959 | 714 | 665 | 546 | 476 | 350 | 259 | 210 | 147 | 70 |
| 0.8 | 1096 | 816 | 760 | 624 | 544 | 400 | 296 | 240 | 168 | 80 |
| 0.9 | 1233 | 918 | 855 | 702 | 612 | 450 | 333 | 270 | 189 | 90 |
| 1.0 | 1370 | 1020 | 950 | 780 | 680 | 500 | 370 | 300 | 210 | 100 |
| 1.1 | 1507 | 1122 | 1045 | 858 | 748 | 550 | 407 | 330 | 231 | 110 |
| 1.2 | 1644 | 1224 | 1140 | 936 | 816 | 600 | 444 | 360 | 252 | 120 |
| 1.3 | 1781 | 1326 | 1235 | 1014 | 884 | 650 | 481 | 390 | 273 | 130 |
| 1.4 | 1918 | 1428 | 1330 | 1092 | 952 | 700 | 518 | 420 | 294 | 140 |
| 1.5 | 2055 | 1530 | 1425 | 1170 | 1020 | 750 | 555 | 450 | 315 | 150 |

Currently orally disintegrating tablet or orally dissolving tablet (ODT) are available in the market. They were designed for people suffering for dysphagia who can take the drugs as ODT without water. Now ODT has been used as a new kind of drug delivery system and has been widely accepted. Currently, many drugs are administered as ODT and Table 2 lists few such drugs. Some of the examples of approved orally fast dissolving tablets are—Loratidine, Cetirizine, Cisapride monohydrate, Risperidone, Zolmitriptan, Hyoscyamine Sulfate, Famotidine, Tramadol HCL, Phloroglucinol Hydrate etc. It is evident from Table 2 that ODT's are useful to various age groups. The same drugs and many additional drugs can also be delivered using ODC technology.

TABLE 2
List of active pharmaceuticals administered as ODT.

| Intended Age | Active ingredient |
|---|---|
| 0-5 years | Clonazepam, Iamotrigine, Hyoscyamine, Iansoprazole, |
| 5-10 years | Aripriprazole, Amphetamine, Fexofanadine, |
| | Desmopressin, Tramadol |
| 10-15 years | Diphenhydramine, Domperidone, Risperidone, Ibuprofen |
| 15-20 years | Meloxicam, Tramadol, |
| Adults | Selegiline, Olanzapine, Metoclopramide, Famotidine |

U.S. Pat. No. 8,105,625 patent describes the formulation of fast dissolving capsules to enhance the therapeutic effect. The capsule comprises of pullulan, a plasticizer, and a dissolution enhancing agent. The capsules have a normalized breakthrough of less than 30 seconds in water at 37° C. EP20080745143 claims the formulation of fast orally dissolving capsules comprising pullulan, one or more plasticizer and a dissolution enhancing agent. The same inventors filed these two patents.

US 2008/0274187 A1 prepared hard capsule compositions comprising carrageenan, locust bean gum, xanthan gum, sorbitol, and pullulan. These capsules eliminated the problem of cracking, embrittlement, chipping and deformation due to water loss and mechanical stress.

Ronald Bodmeier et al. (Int. J. of Pharm.; Vol. 303, 2005, Pages 62-71) prepared fast dissolving capsules for administration in the oral cavity. Their study suggested that the disintegrating time of films decreased with decreasing bloom strength and could be further decreased by addition of sugars or PEGs. Later, they prepared modified conventional hard gelatin capsules either by perforation or by vacuum-drying of conventional hard capsules (Eur. J. Pharma. & Biopharm., Vol. 62, 2006, Pages 178-184). The disintegration time of conventional hard gelatin capsules (HGC) was reduced from 91 to 39 seconds by introducing 6-10 small holes (diameter=25-50 μm) into the capsule shell.

The present patent application proposed slow-release orally dissolving capsules (ODC), which would release the drug over a long time (not a fast dissolving system but a sustained release) and one can administer a drug dosage form without need of water.

### SUMMARY OF THE INVENTION

The present invention proposes a method for the slow release of drugs from ODCs. These ODCs can be administration without coadministration of water or any other liquid. A capsule is one of the solid dosage forms, which is used as a delivery system for an active moiety or moieties. In general, capsules are prepared with a suitable material or matrix such as gelatin, HPMC or another polymer to form a capsule shell. They are administered with water via oral cavity.

The key objective of the invention is to enable patient to take a capsule—ODC, without water or any other liquid. It is not mandatory to administer ODCs without water, but the patients obtain many advantages from the release of drug in the mouth cavity over time. For elderly patients, it is customary to take medication in the evening or before going to bed. If the medications are taken with water,the water intake so late at night may make them use the restroom at night and thereby, disturbing their sleep. In such instances, taking medication without water can be very useful.

Another objective of the invention is to provide a delivery system, which will enable a delivery of large quantity of doses. Many patients such as children, women, old people can't gulp large capsules and tablets. One can administer big capsules such as 00 or 000 as ODC. Based on Table 1, one will be able to deliver 1 to 2 grams of granules with the ODC.

Another objective of the invention is to release of active ingredient(s) in oral cavity to allow buccal and sublingual absorption (or oral transmucosal absorption) and thereby, reduce hepatic first pass effect at least for a portion of the drug which is absorbed through the buccal cavity.

In another embodiment, sterilized ODC are prepared and can be administered by a non-oral route. These will be termed as “dissolving capsules” instead of “orally dissolving capsules”. Polymeric ODC are suitable ionizing radiation sterilization. When the ODCs are prepared under aseptic conditions. ODCs filled with suitable drug can also be placed in the body cavity or under the skin, in the interior wounds etc. where the shell dissolves over time releasing the medicament. A body cavity is any fluid-filled space in the body other than those of blood vessels and lymph vessels. Human body cavities may include—cranial, dorsal, thoracic, ventral, spinal, pelvic. abdominal and abdominopelvic.

The main objective of the invention is to provide a method of administration of ODC to release the active ingredient(s) over a long time allowing a sustained release in those instances where a local action in the mouth cavity is intended. It will also help buccal absorption of drug over longer period.

The present invention is directed to a novel drug delivery system for ODC, comprising a hard-capsule shell which may contain a sweetener, a sequestering agent, suitable salts, a gelling agent and a flavoring agent or combination thereof. The invention teaches the method of administration of capsule. The capsule shell dissolves slowly in the mouth cavity allowing the release of drug composition filled inside the capsule. The sweeteners and flavoring agents will provide an acceptable taste or mask the taste of the drug, if necessary. The sequestering agent or other salts may induce salivation. Many people suffer from dry mouth and saliva will help to swallow the drug particles.

In another embodiment, the sweetener and/or the flavoring agent are coated on the capsules.

The capsules are prepared by a pin-dip method.

In other embodiment of the invention, ODCs further comprising a drug or combination of drugs as microspheres, beads, powder, granules, pellets, mini-tablets, paste, combination thereof as core fill.

In another embodiment of the invention, capsule filling material such as microspheres, granules, beads, pellets, and mini-tablets, can be coated with flavoring agent and/or a sweetener.

In another embodiment of the invention, capsule filling material such as microspheres, granules, beads, pellets, mini-tablets can be coated with polymer or coating material for slow release, sustained release, time-release, controlled release or modified release action or as an enteric coat.

In another embodiment, the drug(s) may be dissolved or suspended in the polymer solution to be used to make the HPMC capsules. This way, the empty capsule shell matrix will have the drug embedded in it. One can use these capsules as ODC.

### DETAIL DESCRIPTION

Definition of Terms Used

The term “orally dissolving capsule” (ODC) refers to a capsule that dissolves in the oral cavity of a patient after getting wet with saliva without need of drinking liquids/water. The term “orally” means the dosage form is administered by mouth. The term “dissolving capsule” means the capsule material is incorporated into a liquid or it dissolves so as to form a solution. The term “Orally Dissolving Capsule” means the orally administered capsule dissolving in the mouth cavity. When the capsule shell dissolves in the mouth, the material filled inside the capsule gets released in the mouth cavity. The ODCs can be administered orally without aid of water or any other liquid. However, the ODCs can behave like a typical capsule and can also be swallowed with water or any other liquid. There are two time points defined in this application—1. Time for the first release of drug—when one feels the first release of drug in the mouth. If the drug does not have any taste, some indicator such as a sweetener can he included in the core of the capsule along with the drug and 2. Time to dissolve the entire capsule—when all the drug from the capsule is released by a complete dissolution of capsule shell.

As the name suggest, the slow-release ODC releases the drug incorporated in the capsule at a slower rate. Typically, the drug will be released from this ODC from 3 minutes to 60 minutes in the mouth cavity. The time for the first release of drug will be over 3 minutes arid the time to dissolve the entire capsule will be up to 60 minutes.

The “empty, hard capsule shell”—as name suggests, it is the capsule shell, which is empty, hard, durable and smooth. It retains its shape and it is dry in nature. As evident from the word “empty”, there is nothing inside core portion of the capsule shell. The capsule shell is prepared using a film-forming composition/matrix. The hard-capsule shell comprises of two parts—a body and a cap. The shorter piece is called the “cap”, which fits over the open end of the longer piece, called the “body”. The body and the cap cooperatively define a hollow capsule. A manufacturer of empty capsule shells sells these to a manufacturer of a drug product who fills up the active drug along with excipients in the capsule shell core.

The term “slow-release ODC” is different from rapid or fast-release ODC. “Rapid/Fast disintegration/dissolution” as used herein should be understood to encompass disintegration/dissolution of at least 80% of the core composition of the ODC, typically 90% and more typically 100% of the core composition in an aqueous medium or in saliva (in the oral cavity) within 10 seconds and at times, even within 5 to 9 second. Rapid/fast ODC is meant to release the drug very fast in 10 seconds to 20 seconds. The term “slow release” refers as dissolution of at least 80% of the composition of the invention, typically 90% and more typically 100% of the composition in an aqueous medium or in saliva the oral cavity) in more than 3 minutes and within 60 minutes. In this case, the dissolution test of ODC is conducted in the mouth cavity and not in the USP dissolution apparatus, which uses 900 mL volume per vessel. The volume of 900 mL in the USP is not representative of volume in the mouth cavity. Specifically, the ODC, in this case, is placed in the buccal cavity. Buccal cavity is that part of the mouth hounded anteriorly and laterally by the lips and the cheeks, posteriorly and medially by the teeth and/or gums, and above and below by the reflections of the mucosa from the lips and cheeks to the gums. It can be also called the vestibule of mouth. This patent teaches a method to administration of ODC. The ODC can be kept in the middle of the mouth cavity. The ODC will move freely and can be sucked on by the tongue. The patent teaches to place the ODC on the side of the mouth between the cheek and the gums. The ODC is left alone to dissolve slowly and not move in the mouth cavity from right to left or left to right. The ODC may be rotated in its place by tongue, if necessary.

The term “immediate release” refers to an immediate release of medicament from a dosage form. When administered to the gastrointestinal tract, it allows the drug to dissolve in the gastrointestinal contents, with no intention of delaying or prolonging the dissolution or absorption of the drug. The patent is intended for a “slow release” of medicaments from the ODC. As the word suggests, the capsule shell dissolves slowly in the mouth cavity and releases the drug in the core of the capsule slowly.

The term “modified release” refers as the dosage form, which is designed to modify the release of the drug over a given time or after the dosage form reaches the required location. Modified release solid oral dosage forms include both delayed- and extended-release drug products. The term “delayed release” refers as the release of a drug (or drug(s) at a time other than immediate following oral administration. The term “extended release” refers as systems which allow for the drug to be released over prolonged time periods. The term “sustained-release” refers to a system, which slows the rate of release of the active ingredient dissolved or dispersed in the system.

The term “empty shell capsule matrix” refers the material with which the empty capsule shell is prepared. The empty shell capsule matrix is generally a solution of gelatin or polymer such as hydroxypropyl methylcellulose with other excipients from which capsule shell cap or body are made by pin-dip method following by drying. The capsule shell includes both parts of the capsule—cap and the body. The material is filled in the capsule body and then the cap is placed on the body of the capsule.

The term “capsule core” refers to the inside empty portion or the cavity within the capsule where other materials such as powders, granules, beads, mini-tablets etc. are filled in. These materials are loaded with drug(s). In another embodiment, a small capsule tilled with one or more drugs is placed in a bigger capsule containing powder or granules of another drug. This way, one can achieve physical separation of active moieties and can prevent drug-drug interactions.

The term “non-aqueous” refers to a system in which the dispersing or dissolving solvent is something other than water. For this patent, the definition is broader encompassing cosolvents similar to water including glycerin, propylene glycol, and polyethylene glycol in pure form or a mixture of water to one or more of these cosolvents.

The term “controlled release” refers to systems which offer a sustained-release profile, but controlled-release systems are actually controlling the drug concentration in the body, not just the release of the drug from the dosage form, as is the case in a sustained-release system.

The term “coating” is defined as a layer of a substance spread over a surface for the protection or decoration and it might be flavored or sweetened.

A “medicament” is an agent that promotes recovery from an ailment or an injury. Similar words to medicament are medicines, drugs, therapeutic agent, biologically active molecule/agent, active component, and an active moiety. These agents affect physical and/or biochemical properties of a biological system. The classes of medicament applicable in this invention include, but not limited to, anti-tumor agents, cardiovascular drugs, hormones, growth factors, steroidal agents, anti-viral agents, antibiotics and the like. The medicament when mixed with suitable excipients forms a drug composition, which is then converted to dosage forms such as microspheres, granules, pellets, mini-tablets, etc. It is assumed, or it imperative that the drug composition in this patent contains a “therapeutically effective amount” of drug(s).

A “sweetener” is a substance that provides a sweetening effect. Sweetener is one especially other than sugar.

The term “sequestering agent” or “chelating agent” refers to a substance that can bind with metal ions to form chelate complex. The sequestering agent can promote salivation by eliminating water hardness. Salt, buffer acids, lemons, cinnamon etc. can also help salivation.

The term “flavoring agent” is defined as the substance that added to give a taste.

The “drug composition” for this patent application means a mixture in which the the therapeutically effective amount of drug(s) is mixed with suitable excipients and the mixture is used as a powder, or converted to granules, pellets, suspension, paste or non-aqueous liquids. An excipient is a substance formulated alongside the active ingredient of a medication included for long-term stabilization, bulking up solid formulations that contain potent active ingredients (thus often referred to as “bulking agents”, “fillers”, or “diluents”), or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerned such as by facilitating powder flowability or non-stick properties, in addition to aiding in vitro stability such as prevention of denaturation or aggregation over the expected shelf life. The “therapeutically effective amount” is the amount of pharmaceutical or nutraceutical medicament needed to treat, totally or partially, a disease state or alleviates one or more symptoms of the condition.

The term “Ayurvedic” medicine refers to one of the world's oldest holistic (“whole-body”) healing systems. It was developed more than 3,000 years ago in India. It's based on the belief that health and wellness depend on a delicate balance between the mind, body, and spirit. Many herbal medicines are being used in the Ayurvedic medical system. The other commonly used system contains—“allopathic” medicines. The allopathic medicine refers to mainstream medical use of pharmacologically active agents or physical interventions to treat or suppress symptoms or pathophysiologic processes of diseases or conditions.

The advantages of slow-release ODCs are—it reduces hepatic first pass effect by allowing absorption of drug from the mouth cavity, dose accuracy in comparison to oral liquids, no need of water or a spoon for administration and lowering of T-max, which is time to achieve the maximum drug concentration in the blood. The hepatic first pass effect or the first pass metabolism is a phenomenon of drug metabolism in the liver whereby the concentration of a drug is greatly reduced before it reaches the systemic circulation. Thus, for drugs which can be absorbed from the buccal cavity, we can achieve lower Tmax and higher AUC or we can reduce the dose of the drug to achieve same pharmacological effect.

Gelatin and hydroxypropyl methylcellulose (HPMC) are the widely used to prepare capsule shells. HPMC capsule shells demonstrate lack of brittleness even at moisture levels below 2%, no cross-linking and improved chemical stability as compared to the gelatin capsule shells. Gelatin capsules have a unique flavor which many people don't like. Gelatin is obtained from animal source and many people do not prefer these capsules. Gelatin does not dissolve easily in mouth and tend to form a lump. We therefore prefer to use polymer capsules in this patent. The polymers that can be used in making the present empty, hard capsule shells can be divided into the following groups: 1) Cellulose or cellulose compounds, which include, but are not limited to, cellulose, cellulose ether, methyl cellulose (MC), HPMC, hydroxypropyl cellulose (HPC), hydroxyethyl cellulose, carboxymethyl cellulose (CMC), cellulose acetate phthalate (CAP), 2) starch-based compounds, which include, but not limited to hydroxyethyl starch, hydroxypropyl starch, hydroxyethyl methyl starch, 3) carrageenans—kappa and iota, 4) Acrylate compounds, which include, but not limited to, polyacrylate, polymethylacrylate, poly(acrylate-methylacrylate), poly(methylacrylate-methyl methacrylate), 5) polyolefins, which include, but limited to, polyvinyl chloride, polyvinyl alcohol, and polyvinyl acetate and 6) pullulan (a polysaccharide polymer consisting of maltotriose units). A hard-capsule dosage form is manufactured by filling the core of the hard-capsule shell (also termed as a “core-fill”) with powders, microspheres, granules, beads, pellets, a tablet, suspension, paste or another capsule (also termed as “inner capsule”). The inner capsule is defined as a capsule filled with a drug composition which is small enough to fit in the orally dissolving outside capsule. In the recent times, scientists have started filling the hard-capsule core with non-aqueous liquids too such oily and/or waxy substances. Currently, the hard-capsule shells are mostly prepared with either gelatin or HPMC. The hard-capsule shells are purchased from the capsule suppliers such as Capsugel, Shionogi and Universal capsules. The process of manufacturing of HPMC or gelatin capsules has been fully established. After drying, the capsule shell contains mainly HPMC or gelatin, small percent of water, colors, plasticizers/gelling agent and other excipients. In this patent application, we propose to add other excipients such as a sweetener, flavoring agent etc. to provide a pleasant taste to the capsule shell during the dissolution in the mouth cavity.

Each capsule has two parts—a body and the cap. As mentioned in the background section, several new types of polymeric substances have been used to manufacture empty, hard capsule shells. Apart from the main constituent of the capsule shell being gelatin or polymeric in nature, the shell also contains other excipients such as plasticizers (e.g., polyethylene glycol, sorbitol, glycerol), stabilizers (antimicrobial and antioxidants), colorants (FD&C colors, titanium dioxide, natural dyes including riboflavin, carotenes, turmeric and caramel) and sequestering agents (citric acid, sodium citrate, and ethylenediaminetetraacetic acid (EDTA)).

In one embodiment citric acid as sequestering agent have been added in the capsule shell matrix. In yet another embodiment, a sweetener is added in hard capsule shell matrix. In yet another embodiment, a colorant is added in hard capsule shell matrix. It yet another embodiment, a flavoring agent is added in the hard-capsule shell matrix.

In one embodiment, different medicaments are incorporated in the hard-capsule core. In another embodiment, a combination of medicaments is incorporated in the hard-capsule core.

Capsule Fill Formulations

The drug composition is defined as the composition containing active drug substances along with suitable excipients. It is then converted to a suitable dosage form such as—powder, granules, pellets, microspheres, mini-tablets, an inner capsule, a non-aqueous suspension, a paste, non-aqueous solution or a combination of two or more. The key is to ensure masking of the taste of the actives, if the taste is repulsive. For the purpose of this patent, the drug composition is filled in the ODC shell, which is prepared by putting together a cap and a body.

The capsule fill formulation comprises one or more drugs or pharmaceutical agents and one or more excipients. Exemplary pharmaceutical agents used in the capsule fill formulation are selected from one or more of, but not limited to:

Selective serotonin reuptake inhibitors such as Fluoxetine, sertraline, paroxetine, fluvoxamine, citalopram, alaproclate and the like

Anti-emetics such as Ondansetron, granisetron, palonosetron, dronabinol, aprepitant, ramosetron, metopimazine, nabilone, tropisetron, metoclopramide, prochlorperazine, trimethobenzamide, dimenhydrinate, prochlorperazine, dolasetron and the like

5HT3 antagonists such as alosetron, ondansetron, granisetron, palonosetron, ramosetron, tropisetron and the like

Anti-epileptics such as carbamazepine, clonazepam, diazepam, divalproex sodium, fosphenyloin, gabapentin, lamotrigine, levetiracetam, oxcarbazepine, phenyloin, pregabalin, primidone, tiagabine, topiramate, valproate sodium, vigabatrin, zonisamide and like

Anti-migraines such as Almotriptan, dihydroergotamine mesylate, eletriptan, frovatriptan, naratriptan, rizatriptan, sumatriptan, zolmitriptan and the like.

Antihistamines, such as diphenhydramine, dirnenhydrinate, perphenazine, triprolidine, pyrilamine, chlorcyclizine, promethazine, carbinoxamine, tripelennamine, brompheniramine, hydroxyzine, cyclizine, meclizitle, clorprenaline, terfenadine, and chlorpheniramine, and the like. Other antihistamines are represented by, but are not limited to, cimetidine, ranitidine, diphenydramine, prylamine, promethazine, chlorpheniramine, chlorcyclizine, terfenadine, carbinoxamine maleate, clemastine fumarate, diphenhydramine hydrochloride, dimenhydrinate, prilamine maleate, tripelennamine hydrochloride, tripelennamine citrate, chlorpheniramine maleate, brompheniramine maleate, hydroxyzine pamoate, hydroxyzine hydrochloride, cyclizine lactate, cyclizine hydrochloride, meclizine hydrochloride, acrivastine, cetirizine hydrochloride, astemizole, levocabastine hydrochloride, and loratadine;

Antacids, such as cimetidine, ranitidine, nizatidine, famotidine, omeprazole, bismuth antacids, metronidazole antacids, tetracycline antacids, clarithromycin antacids, hydroxides of aluminum, magnesium, sodium bicarbonates, calcium bicarbonate and other carbonates, silicates, and phosphates;

Dopamine D1 and D2 antagonists such as Amisulpride, bromperidol, cabergoline, domperidone, fenoldopam, haloperidol, metoclopramide, metopimazine, pergolide mesylate, prochlorperazine, quetiapine, ropinirole hydrochloride, sulpiride, tiapride and zotepine. Nootropics Almitrine dimesylate and raubasine, cevimeline hydrochloride, codergocrine mesylate, donepezil, galantamine, ginkgo biloba extract (EGb 761), memantine, nicergoline, piracetam, rivastigmine, sulbutiamine, tacrine, and vinpocetine.

Statins such as Atorvastatin, cerivastatin, fluvastatin, lovastatin, pitavastatin, pravastatin, rosuvastatin, and simvastatin.

Oral contraceptives such as ethinyl estradiol, norgestrel, norethindrone, ethinodiol, levonorgestrel, mestranol, desogestrel, and norgestimate.

There are many drugs which are known to get absorbed buccally. Table 3 lists some of the marketed products known to provide buccal or sublingual absorption of drugs. Such known drugs can be incorporated in the ODCs for oral transmucosal absorption. Oral transmucosal encompasses both buccal and sublingual absorption of drugs.

TABLE 3
List of representative drugs available as sublingual tablets for buccal
absorption

| Drug | Manufacturer |
|---|---|
| Acyclovir (SITAVIG ®) | Epi Healthcare |
| Nitroglycerin (NITROSTAT ®) | Pfizer |
| Isosorbide dinitrate | Multiple manufacturers |
| Fentanyl citrate (ABSTRAL ®) | Galena Biopharma |
| Buprenorphine hydrochloride | Multiple manufacturers |
| Ergotamine tartrate (ERGOMAR ®) | Rosedale Therapeutic |
| Ergoloid mesylates | Watson |
| Asenapine (SAPHRIS ®) | Merck Sharp & Dohme |
| Miconazole (ORAVIG ®) | Midatech Pharma |
| Nicotine (NICORRETTE ®) | Glaxo-Smith-Klein |
| Buprenorphine hydrochloride and | Multiple manufacturers |
| naloxone hydrochloride | |
| Testosterone (STRIANT ®) | Ausilium Pharma |
| Zolpidem tartrate (INTERMEZZO ®) | Purdue Pharma |

In one aspect, the hard-capsule shell fill formulation optionally further comprises one or more flavoring agents. The flavoring agents that may be used include those flavors known to the skilled artisan, such as natural and artificial flavors. Several flavoring agents are described in U.S. Pat. No. 8,900,629, which are incorporated herein by reference. These flavoring agents may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also, useful flavorings are artificial, natural and synthetic fruit flavors such as vanilla, and citrus oils including, without limitation, lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixture. Commonly used flavors include mints such as peppermint, menthol, artificial vanilla, cinnamon derivatives, and various fruit flavors, whether employed individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. The flavor is present from about 0 to about 5% by weight of the capsule shell fill formulation. In one embodiment, the flavor is not incorporated in the capsule shell matrix, but rather spread on the capsule shells after the capsule cores are filled with the drug formulation.

In one aspect, the hard-capsule shell fill formulation optionally further comprises one or more saliva stimulating agents. One or more salivary stimulants can be optionally added to the capsule shell fill formulation. Several salivary stimulants are described in U.S. Pat. No. 8,900,629, which are incorporated herein by reference. Salivary stimulants include, but are not limited to, certain organic acids, and sweeteners. Organic acid salivary stimulants include adipic, ascorbic, citric, fumaric, lactic, malic and tartaric acids. Preferred organic acids are malic and ascorbic acids. The most common sweeteners for use as saliva stimulating agents are sugars such as glucose, dextrose, fructose, lactose, maltose, xylose, sucrose, corn sugar syrup, and other sweet mono- or di-saccharides, as well as artificial sweeteners such as acesulfame, aspartame, saccharin, as well as xylitol and other polyols. Preferred sweeteners that are known to be used as salivary stimulants include maltose, acesulfame, aspartame and saccharin.

In one aspect, the hard-capsule shell fill formulation optionally further comprises one or more colorants and opacifiers. Colorants include such compounds as, by way of example and without limitation, titanium dioxide, talc, FD&C Red No. 3, FD&C Red No. 20, FD&C Yellow No. 6, FD&C Blue No. 2, FD&C Green No. 5, FD&C Orange No. 5, FD&C Red. No. 8, caramel, ferric oxide, other FD&C dyes, lakes, and natural coloring agents such as grape skin extract, beet red powder, beta-carotene, annatto, carmine, turmeric, paprika, and other materials known in the art. The amount of coloring agent used will vary as desired.

The drugs or medicament(s) with suitable excipients are converted to various types of filling materials. Filling material of the capsule such as granules, pellets, powder, suspension, semisolids and inner capsules, can be coated for controlled release or modified release of active ingredient. Several coating agents are described in U.S. Pat. No. 7,713,550 B2, which are incorporated herein by reference.

in certain embodiments of the present invention, wherein the dosage form contains a pharmaceutically acceptable polymer, the pharmaceutically acceptable polymer is for example and without limitation, HPC, HPMC, MC, CMC, vinyl acetate/crotonic acid copolymers, maleic anhydride/methyl vinyl ether copolymers, polyalkylene oxide including but not limited to polyethylene) oxide, poly(methylene oxide), polybutylene oxide); poly(hydroxy alkyl methacrylate); poly(vinyl) alcohol, having a low acetal residue, which is cross-linked with glyoxal, formaldehyde or glutaraldehyde and having a degree of polymerization of from 200 to 30,000; a mixture of methyl cellulose, cross-linked agar and carboxymethyl cellulose; a hydrogel forming copolymer produced by thrilling a dispersion of a finely divided copolymer of maleic anhydride with styrene, ethylene, propylene, butylene or isobutylene cross-linked with from 0.001 to 0.5 moles of saturated cross-linking agent per mole of maleic anhydride in the copolymer; CARBOPOL® acidic carboxy polymers having a molecular weight of 450,000 to 4,000,000; CYANAMER® polyacrylamides; cross-linked water swellable indenemaleic anhydride polymers; GOODRITE® polyacrylic acid having a molecular weight of 80,000 to 200,000; starch graft copolymers; AQUA-KEEPS® acrylate polymer polysaccharides composed of condensed glucose units such as diester cross-linked polyglucan and the like. Other polymers, which form hydrogels are described in U.S. Pat. Nos. 3,865,108; 4,002,173 and 4,207,893 have been incorporated by reference. Mixtures of the pharmaceutically acceptable polymers may also be used. In certain preferred embodiments the pharmaceutically acceptable polymer in combination with the drug can form a drug matrix for the controlled delivery of the drug.

Other excipients or inactive ingredients such as calcium carbonate, Croscarmellose sodium, Cellulose, Carboxymethylcellulose calcium, Calcium stearate, Castor oil hydrogenated, calcium phosphate di or tri basic, Glyceryl behenate, Glyceryl monostearate, Lactose hydrous or anhydrous or monohydrate or spray dried, Magnesium stearate, Magnesium carbonate, Magnesium oxide, PEG, Polyoxy140 stearate, Starch pregelatinized, Sodium lauryl sulfate, starch, Sodium benzoate, stearic acid, saccharin sodium, Sodium stearyl fumarate, sodium chloride, and talc can be used the ODC formulations.

Method of Slow Release of Drugs From ODCs

This patent teaches a method for slow release of drugs from ODC. The ODC can be kept in the middle of the mouth cavity. The ODC will move freely and can be sucked on by the tongue. The patent teaches to place the ODC on the side of the mouth in the buccal cavity between the cheek and the gums. The ODC is left alone to dissolve slowly and is not moved in the mouth cavity from right to left or left to right. The ODC may be rotated in its place by tongue, if necessary. The patent describes two methods for the administration of ODCs. In Method 1, ODC is placed in the space between the lower guru and the cheek or termed as a buccal cavity. The capsule is moved within the space with the help of tongue intermittently. The capsule, if not moved, softens, swells and sits for a long time (over 15-30 minutes). Moving of capsules in the same place helps to rotate the capsule and bring in some saliva in the cavity. The time for the first release of drug is noted. The time for the entire capsule to dissolve is noted too. In Method 2, ODC is placed on the tongue in the middle of the mouth cavity. It was rotated and sucked normally (similar to sucking lozenges). The time for the first release of drug is noted. The time for the entire capsule to dissolve is noted too. The drug formulation (powder, granules etc.) may not have any taste and it may be hard to determine the time for the first release of the drug. In that case, time to sense the release of the sweetener or the flavor is noted and considered as the time for the first release of drug.

Examples of Various ODC Formulations

With the following examples, one skilled in the art, can understand and use the present invention. Many more formulations can be developed using the ODC technology.

### EXAMPLE 1

Loratidine 5 mg Capsule for Allergy Symptoms

A capsule composition containing HPMC is prepared by incorporating a sweetener, Acesulfame-K and citric acid as sequestering agent. The capsules are prepared using the pin-dip method. Capsules are also prepared with HPMC only or HPMC and sweetener optionally or HPMC and sequestering agent or combination of thereof.

Capsule fill formulation containing Loratidine equivalent to 5 mg dose is filled in size ‘3’ hard capsule core. Powder containing Loratidine is prepared by trituration to produce the same particle size and then mixed with excipients by geometric dilution method. Formula for Loratidine powder is given below:

TABLE 4
A typical composition of Loratidine
orally dissolving capsule fill formulation

| Ingredient | Quantity (mg) |
|---|---|
| Loratidine | 5 |
| Corn starch | 20 |
| Lactose | 150 |
| Magnesium stearate | 5 |
| FD&C Blue | 1 |

### EXAMPLE 2

Simvastatin 40 mg Capsules for Cholesterol

Granules containing Simvastatin are prepared by using a conventional technique. Simvastatin equivalent to 40 mg dose are filled in size ‘0’ HPMC hard capsule core. Formula for granule preparation of simvastatin is given below. A similar composition can also be used for different strengths of simvastatin or other statins.

TABLE 5
A typical composition of simvastatin
orally dissolving capsule fill formulation

| Ingredient | Quantity (mg) |
|---|---|
| Simvastatin | 40 |
| Lactose anhydrous | 332 |
| Microcrystalline cellulose | 8 |
| Pregelatinised maize starch | 20 |
| Butylhydroxyanisole | 5 |
| Magnesium stearate | 20 |
| Flavoring agent | 10 |

### EXAMPLE 3

Ibuprofen 200 mg Capsule for Pain Relief and to Reduce Fever

Granules of Ibuprofen are prepared by using a conventional technique. Granules containing ibuprofen equivalent to 200 mg dose are filled in size ‘0’ HPMC hard capsule core. Similar composition can be used to prepare different strengths too. Formula for granules of Ibuprofen is given below:

TABLE 6
A typical composition of orally dissolving
ibuprofen capsule fill formulation.

| Ingredient | Quantity (mg) |
|---|---|
| Ibuprofen | 200 |
| Maize starch | 10 |
| Pregelatinized starch | 15 |
| Sorbitol powder | 100 |
| Colloidal silica anhydrous | 20 |
| Stearic acid | 5 |
| Flavoring agent | 10 |

### EXAMPLE 4

Tenofovir 300 mg Capsule for Hepatitis B Treatment

Tenofovir equivalent to 300 mg dose are filled in size ‘00’ hard capsule core. Tenofovir pellets are prepared by using a conventional technique. Pellets are coated with a sweetener. Additionally, capsule may be coated with a flavoring agent. Tenofovir is often administered in combination with other anti-viral agents. The other antiviral agent is either mixed with tenofovir in the same pellet or can be made as an independent pellet or it is tilled in the capsule as granules along with tenofovir pellets. One such combination drug can be Tenofovir/emtricitabine. Formula for preparation of tenofovir pellets is given below:

TABLE 7
A typical composition of tenofovir pellets to be filled
in orally dissolving capsule fill formulation.

| Ingredient | Quantity (mg) |
|---|---|
| Tenofovir | 300 |
| Lactose monohydrate | 250 |
| Pregelatinised starch | 30 |
| Croscarmellose sodium | 65 |
| Microcrystalline cellulose | 10 |
| Flavoring agent | 25 |
| Magnesium stearate | 12 |
| Sweetener | 30 |

### EXAMPLE 5

Capsule in Capsule formulation: Ibuprofen 200 mg/Pseudoephedrine HCl 30 mg Capsule for Cold and Sinus

Preparation of an inner capsule: Pseudoephedrine equivalent to 30 mg dose are filled in size ‘5’ hard capsule core. Powder containing Pseudoephedrine is prepared by trituration method to produce the same particle size and then mixed by geometric dilution method. Formula for Pseudoephedrine powder is given below. The inner capsule of pseudoephedrine (size 5) is filled in the orally dissolving capsule along with ibuprofen granules.

TABLE 8
A typical composition of pseudoephedrine powder

| Ingredient | Quantity (mg) |
|---|---|
| Pseudoephedrine | 30 |
| Cellactose | 24 |
| Colloidal anhydrous silica | 2 |
| Flavoring agent | 3 |
| Magnesium stearate | 1 |

Preparation of ibuprofen granules: Granules of Ibuprofen are prepared by using a conventional technique. Ibuprofen granules containing equivalent to 200 mg dose are filled in size ‘00’ HPMC hard capsule core with pseudoephedrine capsule. Formula for granules of Ibuprofen is given below:

TABLE 9
A typical composition of ibuprofen granules to be filled in an
orally dissolving capsule along with pseudoephedrine inner capsules.

| Ingredient | Quantity (mg) |
|---|---|
| Ibuprofen | 200 |
| Sorbitol powder | 40 |
| Pregelatinized starch | 30 |
| Colloidal silica anhydrous | 10 |
| Stearic acid | 5 |
| Flavoring agent | 10 |

### EXAMPLE 6

Ayurvedic Capsules for Mouth Ailments

The Ayurvedic drug composition is defined as the composition containing active Ayurvedic substances along with suitable excipients. It is then converted to a suitable dosage form such as—powder, granules, microspheres, pellets, mini-tablets, a non-aqueous suspension, a paste, non-aqueous solution or a combination of two or more.

TABLE 10
Typical composition of an Ayurvedic orally dissolving capsule

| Ingredient | Quantity, mg |
|---|---|
| Turmeric powder | 200 |
| Eucalyptus oil | 4 |
| Clove oil | 4 |
| Peppermint oil | 4 |
| Jeshtimadh | 100 |
| Sorbitol | 100 |

The oil components are first mixed as a solution and loaded on the mixture of solid components to provide a free-flowing powder, which is filled in the capsules.

Method of Dissolution of Capsules in the Mouth

Three subjects were administered the Ayurvedic turmeric capsules. Two types of methods were used to administer ODCs. In Method 1, the subjects placed the capsule in the space between the lower gum and the cheek. The capsule was moved within the space intermittently with the help of tongue. The capsule, if not moved, softens, swells and sits for a long time (over 25-30 minutes). Moving of capsules helps to rotate the capsule and bring in some saliva in the cavity. The time for the first release of drug was noted. The time for the entire capsule to dissolve was noted too. In Method 2, the capsule was placed on the tongue in the middle of the mouth cavity. It was rotated and sucked normally. The time for the first release of drug was noted. The time for the entire capsule to dissolve was noted too. The product contained three essential oils and it was easy to determine the release of powder from the capsules. Table 11 lists the time to release 1^{st} drug and time to dissolve the entire capsule. It also states the mean values for each subject. In another experiment, these capsules (n=3) were dropped with sinkers in the USP I dissolution apparatus in water at 37 deg C. The dissolution medium was rotated at 75 rpm. The capsules did not dissolve in 3 hours. It is clear that HPMC, even though has high water solubility, faints a gel in water. There was a layer of gel on the capsule shell. The gel took a long time to dissolve in water.

TABLE 11
Inter- and intra-subject variation in the dissolution of orally dissolving
capsules of the ayurvedic formulation of turmeric using two methods of administration

| | Subject 1 | | Subject 2 | | Subject 3 | |
|---|---|---|---|---|---|---|
| | Time for | Time to | Time for | Time to | Time for | Time to |
| | the first | dissolve | the first | dissolve | the first | dissolve |
| | release | entire | release | entire | release | entire |
| Capsule # | of drug | capsule | of drug | capsule | of drug | capsule |

Method of administration - The capsule was placed between the gum and the cheek. It was
moved in the same space intermittently.

| | | | | | | |
|---|---|---|---|---|---|---|
| Capsule 1 | 4 min 55 sec | 12 min | 2 min 21 sec | 8 min 50 sec | 3 min 57 sec | 12 min |
| | | 18 sec | | | | 51 sec |
| Capsule 2 | 4 min 3 sec | 14 min | 3 min 6 sec | 9 min 12 sec | 3 min 27 sec | 14 min 5 sec |
| | | 13 sec | | | | |
| Capsule 3 | 5 min 53 sec | 10 min | 3 min 50 sec | 8 min 5 sec | 4 min 27 sec | 14 min |
| | | 27 sec. | | | | 23 sec |
| Mean ± SD | 295 ± 55 | 739 ± 113 | 186 ± 44 | 522 ± 34 | 237 ± 30 | 834 ± 54 |
| (sec) | | | | | | |

Grand mean values using Method 1:
Time for the first release of drug: 239 ± 62 sec (% RSD - 25.7%)
Time for the entire capsule to dissolve: 698 ± 153 sec (% RSD - 21.9%)

Method of administration #2 - The capsule was placed in the mouth cavity and was
allowed to dissolve by moving within the cavity

| | | | | | | |
|---|---|---|---|---|---|---|
| Capsule 1 | 47 sec | 3 min 37 sec | 43 sec | 3 min 2 sec | 55 sec | 5 min 10 sec |
| Capsule 2 | 42 sec | 4 min 1 sec | 41 sec | 2 min 55 sec | 59 sec | 4 min 57 sec |
| Capsule 3 | 43 sec | 2 min 27 sec | 45 sec | 3 min 22 sec | 57 sec | 5 min 56 sec |
| Mean ± SD | 44 ± 3 | 202 ± 49 | 43 ± 2 | 186 ± 14 | 57 ± 2 | 321 ± 31 |
| (sec) | | | | | | |

Grand mean values using Method 2:
Time for the first release of drug - 48 ± 7 sec (% RSD - 14.6%)
Time for the entire capsule to dissolve - 236 ± 70 (% RSD - 29.8%)

In the mouth cavity, when the capsule is kept in the middle of the mouth cavity, the capsule is moved around by the tongue. The movement and frictional force helps to dissolve the gel of HPMC capsule surface and the capsule dissolves very quickly. In Method 2, the time for the first release of drug was less than 1 minute (grand mean—48±7 sec, n=9). Time for the entire capsule to dissolve was 236±70 seconds. The same capsule, when kept between the gum and the cheek in Method 1, the grand time for the first drug to release was 239±62 seconds. The grand time to release the entire capsule was 698±153 seconds (n=9). In both methods, there is a significant intra- and inter-subject variability observed. Still, the times observed using Method 1 were significantly greater than times observed using Method 2. Thus, Method 1 released the drug significantly slowly compared to Method 2. The time for the dissolution of capsule in mouth cavity was much shorter compared to the time needed for the capsule to dissolve in the USP 1 dissolution apparatus. The polymer forms a gel, in the mouth cavity, the top layer of gel is taken away due to frictional action. Thus, the in vitro dissolution test did not predict the dissolution in the mouth cavity. We want to attain a slow release of drug in the mouth cavity so as to get a local effect and may have oral submucosal absorption of the drug. Thus, we are claiming Method 1 as the method of administration of the ODC to provide much slower release of drug compared to observed by Roger et. al, (U.S. Pat. No. 8,900,629). Roger et al. did not teach this method in their patent application.

### EXAMPLE 7

Cetirizine 10 mg Extended Release Capsule for Allergy Symptoms

Cetirizine beads are filled in size ‘2’ HPMC hard capsule core. Cetirizine beads are prepared by using a conventional technique and coated with HPMC solution. Formula for the cetirizine beads is given below:

TABLE 12
A typical composition of cetirizine beads
filled in orally dissolving capsules

| Ingredient | Quantity (mg) |
|---|---|
| Cetirizine | 10 |
| Lactose monohydrate | 150 |
| Starch, corn | 15 |
| Talc | 10 |
| Povidone K 30 | 50 |
| Flavoring agent | 10 |
| Magnesium stearate | 12 |
| Sweetener | 10 |

### EXAMPLE 8

ODC With Ethinyl Estradiol in the Cap and Norgestrel in the Body and No Drug Composition in the Capsule Core

The base composition of cap and body of the ODC contained HPMC, water, carrageenan, potassium ions, a sweetener, and a flavoring agent. The composition from which caps for the capsules are made contains ethinyl-estradiol. The amount added in the solution from which the Caps are made is chosen in such a way that after drying each Cap contains 50 micrograms of ethinyl estradiol. The composition from which Body for the capsules are made contains norgestrel. The amount of norgestrel added in the HPMC solution from which the Body are made is chosen in such a way that after drying each body contains 500 micrograms of norgestrel. Then the cap and the body are put together, the empty capsule shell contains 50 micrograms of ethinyl estradiol in the Cap matrix and 500 micrograms of norgestrel in the Body matrix. The empty capsule shells are not filled with any other drug and are administered as ODC. One can also have ODCs with 30 micrograms of ethinyl estradiol and 300 micrograms of norgestrel. There are various combinations of contraceptive products already available in the market. This concept can be applied to any combination. For example, in another ODC, the cap contains 0.02 mg ethinyl estradiol and the body contains 3 mg of drospirenone. It is not just the contraceptives, but other drug combinations can be delivered as ODC. The key is—the dose of each drug should be very small, less than 5 mg per capsule. In another example, single drug is dissolved in the capsule composition from which Cap and Body of the capsule are made. In another ODC, 3 mg of melatonin, a commonly used sleep-aid agent is incorporated in the ODC shell matrix.

Summary

From the foregoing, it will be seen that this invention opens several possibilities using the polymeric hard capsules containing different medicaments along with a combination of drugs in the core. As described in the patent application, these capsules are to be placed in the mouth, specifically between the gum and the cheeks. The ODC should be rotated in place intermittently, but not moved to the middle of the mouth cavity. This will aid to dissolve the capsule slowly. The key objective of this formulation is to achieve a slow release of drugs from the ODC. The capsule shell dissolves in up to 60 minutes releasing the drug filled in the capsule slowly in the mouth cavity. One of the advantages of this delivery system is—the dosage form is administered without the aid of water. A portion of drug may get absorbed through the mucosa of mouth cavity and the remaining portion proceeds to stomach. The portion absorbed from the mouth cavity (oral transmucosal absorption) enters the blood stream and does not undergo first-pass metabolism in the liver. Many drugs may have local action such as the Ayurvedic formulation described in this patent specification. As ODCs dissolve in the mouth cavity, large capsules can also be administered, such as size ‘000’ which are not used in the pharmaceutical drug delivery system. In another embodiment, drug is incorporated in the capsule shell matrix of ODC and no drug is filled in the capsule core. Polymeric ODC are suitable ionizing radiation sterilization. When the ODCs are prepared under aseptic conditions, ODCs filled with suitable drug can also be placed in the body cavity or under the skin, in the interior wounds etc. where the shell will dissolve over time releasing the medicament.

While specific examples have been presented here, various modifications can be made and the invention is not limited to the examples shown in this patent application.

What is claimed:

1. A method for a slow release of drug(s) from an orally dissolving capsule comprising: (a) filling therapeutic dose of a drug(s) as a drug composition in a non-gelatin polymeric hard shell capsule; (b) placing the capsule in the buccal cavity between the gum and the cheek; (c) allowing it dissolve slowly on its own in saliva without any additional fluid and (d) maintaining the capsule in the buccal cavity between the cheek and the gum, moving in place intermittently until the capsule is completely dissolved slowly releasing the drug up to 60 minutes to allow partial or full oral transmucosal drug absorption; wherein the capsule comprises a cap and body, each of the cap and body comprising a polymer.

2. A method for a slow release of drug(s) from an orally dissolving capsule as in claim 1 in which the polymer is selected from hydroxypropyl methylcellulose, hydroxypropyl cellulose, methyl cellulose and mixtures thereof.

3. A method for a slow release of drug(s) from an orally dissolving capsule in claim 1, comprising: a body and a cap prepared using hydroxypropyl methylcellulose polymer as one of the components.

4. A method for a slow release of drug(s) from an orally dissolving capsule as in claim 1 wherein the drug composition in the core-fill is: a powder or granules or microspheres or pellets or mini-tablets or an inner capsule or a non-aqueous suspension or a paste or a non-aqueous solution or combination of two or more.

5. A method for a slow release of a drug(s) from an orally dissolving capsule comprising: (a) filling therapeutic dose of a drug(s) as a drug composition in a non-gelatin polymeric hard shell capsule containing a sweetener, a sequestering agent, optionally a flavoring agent, optionally a gelling agent; (b) placing the capsule patient in the buccal cavity between the gum and the cheek; (c) allowing it dissolve slowly on its own in saliva without any additional fluid and (d) maintaining the capsule in the buccal cavity between the cheek and the gum, moving in place intermittently until the capsule is completely dissolved slowly releasing the drug up to 60 minutes; wherein the capsule comprises a cap and body, each of the cap and body comprising a polymer.

6. A method for a slow release of a drug(s) from a sterilized dissolving capsule comprising (a) filling therapeutic dose of a drug(s) as a drug composition in a non-gelatin polymeric hard shell capsule under aseptic conditions; (b) sterilizing filled capsules by radiation; (c) placing the capsule in the body cavity or under the skin or in the wound; (d) allowing it to dissolve slowly on its own in the body fluid without any additional fluid and (e) maintaining the capsule in place until the capsule is completely dissolved slowly releasing the drug; wherein the capsule comprises a cap and body, each of the cap and body comprising a polymer.

7. A method for a slow release of a drug(s) from an orally dissolving capsule as in claim 5 and claim 6 wherein the drug composition in the core-fill is: a powder or granules or microspheres or pellets or mini-tablets or an inner capsule or a non-aqueous suspension or a paste or a non-aqueous solution or combination of two or more as.

8. A method for a slow release of a drug(s) from an orally dissolving capsule as in claim 5 and claim 6 in which the polymer is selected from hydroxypropyl methylcellulose, hydroxypropyl cellulose, methyl cellulose and mixtures thereof.

9. A method for a slow release of a drug(s) from an orally dissolving capsule as in claim 1, and claim 5, wherein the capsule is selected from a group consisting of size 5, size 4, size 3, size 2, size 1, size 0, size 0e1, size 00, size 00e1, and size 000.

10. A method for a slow release of a drug(s) from a dissolving capsule as in claim 6, wherein the capsule is selected from a group consisting of size 5, size 4, size 3, size 2, size 1, size 0, size 0e1, size 00, size 00e1, and size 000.

11. A method for a slow release of a drug(s) from an orally dissolving capsule as in claim 1 and claim 5, wherein the drug is dissolved or suspended in the empty capsule shell matrix.

12. A method for a slow release of a drug(s) from a dissolving capsule as in claim 6, wherein the drug is dissolved or suspended in the empty capsule shell matrix.
